(19) United States
(12) Patent Application Publication
(10) Pub. No.: US 2007/0208410 A1
(43) Pub. Date: Sep. 6, 2007

(54) Methods and Apparatus for Treatment of Aneurysms Adjacent to Branch Arteries

(75) Inventors: Humberto Berra, Cooper City, FL (US); Michael T. Wright, Allen, TX (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/743,296

(22) Filed: May 2, 2007

Related U.S. Application Data
(63) Continuation-in-part of application No. PCT/US06/34395, filed on Sep. 1, 2006.
(60) Provisional application No. 60/713,967, filed on Sep. 2, 2005.

(51) Int. Cl. A61F 2/06

(52) U.S. Cl. 623/1.13; 623/1.35

(57) ABSTRACT

Methods and apparatus for delivering and reducing the profile of a catheter for delivering a stent graft including integral branch connections. A configuration of a branch vessel stent graft, including a branch vessel connection connecting a very thin walled PTFE tubular branch to a woven polyester main body. The connection is made by using by overmolding the PTFE on a polymer ring, such as silicone. In another configuration, a main body portion includes branch portions where ends of the branch portions are connected to leads extending from a sheath of a stent graft compressed in a delivery catheter. The leads are routed into accessible branches of body lumens and act as pullwires to pull the branch portions into position in their respective branches as the delivery catheter is released to deploy the main body of the stent graft. Apertures in the main body portion are alignable with the branch lumens and an anchoring stent is separately deployed to extend and/or main the extension of the branch portion in the branch lumen and anchor it therein.

### RELATED APPLICATIONS

This application is a continuation-in-part of PCT/US2006/034395 filed on Sep. 1, 2006; which claims priority to U.S. Provisional Patent Application 60/713,967 filed Sep. 2, 2005, all of which are incorporated herein by reference.

### FIELD OF THE INVENTION

The field of the invention is the treatment of vascular abnormalities. More particularly, the field of the invention is the treatment of vascular abnormalities by the placement of an excluding device to bypass the abnormality, and to seal the abnormality off from access to any fluids passing through the vasculature at the location of the abnormality. More particularly still, the field of the invention relates to the field of the treatment of aneurysmal disease, wherein an exclusion device, such as a stent graft, is deployed across an aneurysmal site in a blood vessel such as an aorta, to exclude blood flow to the aneurysmal sac of the blood vessel and simultaneously provide a conduit for blood flow past the aneurysmal site.

### BACKGROUND OF THE INVENTION

“Thoracic aortic aneurysm” is the term used to describe a condition where a segment of the aorta in the thoracic region is dilated to a diameter greater than its original diameter. Thoracic aortic aneurysms are caused by hardening of the arteries (atherosclerosis), high blood pressure (hypertension), congenital disorders such as Marfan's Syndrome, trauma, or less commonly, syphilis. Atherosclerosis is by far the most common cause. Thoracic aneurysms occur in the ascending aorta (25% of the time), the aortic arch (25% of the time) or the descending thoracic aorta (50% of the time). Where dilation meets or exceeds 50% of the original aortic diameter, i.e., where the diameter of the aorta is 150% of the original or expected diameter of the aorta, intervention generally is deemed necessary. Without intervention, the aneurysm may continue to expand, leading to the possibility of tearing or rupture of the aorta, and likely to death. Intervention includes techniques such as replacement of the aorta with a synthetic lumen (graft) which is sewn to healthy tissue of the aorta where the aneurysmal portion of the aorta is surgically removed or exposed, or, less invasively, by the intravascular placement across the aneurysmal site of an exclusion device, such as a stent graft. The stent graft is a tubular member designed to provide a conduit carrying blood flow through the aorta without allowing the systemic pressure of the blood from the main vessel (the aorta) to further stretch the aneurysm, with the goal of excluding fresh blood from the weakened aneurysmal wall of the aorta. To achieve this result, the stent graft must span the weakened blood vessel wall so that the opposed ends of the stent graft engage and seal against healthy blood vessel or aorta wall tissue.

A stent graft includes a stent framework (stent portion), which provides physical support of the stent graft in a tubular configuration once deployed at a vascular location, and a graft portion, comprising an excluding material, which is sewn or otherwise attached to the stent portion and which provides a relatively fluid-tight conduit for blood flow through the stent graft and past the aneurysm site. Insertion/deployment of a stent graft can be performed without a chest incision using specialized catheters that are introduced through arteries usually at a location in a leg adjacent to the groin.

The thoracic aorta has numerous arterial branches. The arch of the thoracic aorta has three major branches, all of which arise from the convex upper surface of the arch and ascend through the superior thoracic aperture to the root of the neck. The brachiocephalic artery originates anterior to the trachea. The brachiocephalic artery divides into two branches, the right subclavian artery (which supplies blood to the right arm) and the right common carotid artery (which supplies blood to the right side of the head and neck). The left common carotid artery arises from the arch of the aorta just to the left of the origin of the brachiocephalic artery and supplies blood to the left side of the head and neck. The third branch of the aortic arch, the left subclavian artery, originates behind and just to the left of the origin of the left common carotid artery and supplies blood to the left arm. The proximity of an aneurysm to a branch artery, or the involvement of a branch artery opening in the aneurysm, may limit the ability to exclude an aneurysm by the use of an excluding device. An abdominal aortic aneurysm may span a portion of the aorta connected to branch arteries, such as the renal arteries. Where the dilation of the aorta caused by the aneurysm extends into the region of the aorta from which the branch arteries extend, the ends of the stent graft will need to span a branch artery location to enable sealing against healthy aortic wall tissue. Further, where the aneurysm implicates the aorta wall at the branch artery location and thus the aorta is dilated at the branch artery location, their may be a gap between the aorta wall and the stent graft at the branch artery location. Therefore, simply locating an aperture through the exclusion device at the branch artery location to enable blood flow into the branch artery could result in significant blood flow to the aneurysmal sac portion of the aorta.

One mechanism used to provide sealing of an aneurysmal site where the aneurysm implicates a portion of the artery which spans a branch vessel location is to employ an artificial branch lumen as an integral feature of the stent graft, and extend this artificial branch lumen into the branch artery, such that the main body of the stent graft from which it extends can be deployed, at the opposed ends thereof, against healthy aorta tissue, and the artificial branch lumen may extend into, and seal against the wall of, the branch artery. U.S. Pat. No. 6,723,116 by Taheri describes such a configuration. Several such artificial branch lumens may be deployed, such that the stent graft may extend across the location of multiple branch lumens without excluding blood flow to the branch lumens. To ensure that the artificial branch lumens seal against the walls of the branch arteries, and thereby prevent leakage of blood between the artificial branch lumen and the branch artery wall and thence to the aneurysmal region, the artificial branch lumen includes a compressed stent positioned therein prior to delivery of the stent graft to the aneurysmal aorta.

One problem which may be encountered in the deployment of a stent graft is caused by the physiology of the patient within whom the stent graft is to be deployed. To deploy the stent graft, it is typically collapsed or compressed, in a configuration by which it may be expanded at the aneurysmal site to span the aneurysm and seal against the vessel wall on both sides spanning the aneurysm. The inclusion of integral artificial side branch grafts to the stent graft increases the bulk of the stent graft that must be compressed and delivered through a catheter. This usually requires a larger diameter catheter. As the stent graft is delivered intravascularly, the larger the size of collapsed cross-section of the stent graft, the larger the diameter or crossing profile of the delivery sheath and the catheter which is used to carry the stent graft to the deployment location and to deploy the stent graft will require that patients have larger femoral arteries as a prerequisite for them being considered for such an integrated branch attached stent graft. Where the patients' arteries through which the catheter is routed include a restriction, including restrictions caused by disease or by abrupt changes in direction, the crossing profile of the catheter which can pass through the restriction is limited and it may be impossible to deploy a stent graft having artificial branch lumens through such a location, particularly where additional bulk is added to the stent graft to enable continuous stent grafting from the main body of the stent graft into branch vessel location.

Therefore, there is a desire in the art to achieve a greater success of aneurysm repair and applicability and healing, and in particular, by using techniques to provide smaller diameter mechanisms and methods to enable endoluminal repair using such smaller stent grafts or the placement of other exclusion devices across branch vessels while still maintaining flow into those branch vessels adjacent to aneurysmal locations.

### SUMMARY OF THE INVENTION

Embodiments according to the present invention address aneurysm repair and the low profile construction of and placement of an exclusion device to span and seal across an aortic blood vessel's aneurysm while routing blood flow to branch vessels which are also spanned by the exclusion device. Specifically, embodiments according to the present invention provide a construction of a stent graft for use in applications where the stent graft must, to properly exclude the weakened vessel wall caused by aneurysm, span the opening or intersection of a branch vessel with the main vessel, such as in the treatment of abdominal or thoracic aortic aneurysms. The stent graft excludes the aneurysmal region from exposure to fresh blood flow, without blocking or otherwise impeding the flow of blood to arteries that branch off from the abdominal or thoracic aorta. Embodiments according to the invention are readily applicable to uses in aneurysmal locations where branch vessels or other flow lumen discontinuities are present. Additionally, where the aorta is dilated at, or immediately adjacent to, the branch vessel location, embodiments according to the invention enable the use of a stent graft providing a synthetic flow lumen having the circumference or diameter of a healthy aorta through the aneurysmal location. Secondary flow lumens extend from the main body of the stent graft and into the adjacent branch vessels such that the secondary flow lumens span any gap between the main body of the stent graft and the adjacent aorta wall before entry into the branch vessel lumen. Such a configuration provides a contribution to an anchoring force preventing stent graft migration in the aorta, while reducing the crossing profile needed to deliver the exclusion device to the aneurysmal site.

Thus, in one embodiment there is provided an intravascular treatment device, including an aperture alignable with a branch vessel of a body, composed of a generally tubular main body which functions as an exclusion device having at least one aperture therein, and at least one branch vessel flow element disposable in the aperture and extendable into the branch lumen. In a further aspect, the branch vessel flow element integral with the tubular main body is engageable with the branch lumen to allow the aperture from which said branch vessel flow element emanates in the exclusion device to be secured in a desired location with the aperture aligned with, and the branch vessel flow element extending into, the branch lumen. The branch vessel flow element may, in a further aspect, include both a first extendable element, provided with, and deployed integrally with, the tubular main body, and an additional second element, which may be separately deployed to the branch lumen location to provide the securing, and sealing, of the branch vessel flow element into the branch lumen. In one aspect, the exclusion device is a stent graft. In an additional aspect, the branch lumen is a branch artery in the aorta, such as those present as branch arteries in the abdominal or thoracic aorta. In one specific aspect, the apertures may include flange(s) attached to the tubular graft material forming, in part, the main body of the stent graft, which provide side branch openings in the stent graft main body. Such flanges would be provided in combination with a secondary extension member, which is likewise a tubular element, configured to be deployed with the stent graft main body to the aneurysmal location and deployed by extending from each such flange to enable sealing of the branch vessel flow element in and to the branch lumen wall. The flange(s) are thin walled members. A secondary positioning member, such a plurality of leads, is deployed therewith to extend the branch vessel flow elements from the flange outwardly in the direction of the branch lumen location.

In a method for deploying the exclusion device, an exclusion device is provided having a generally tubular main body, through the wall of it is provided at least one aperture and a first member extending outwardly from the tubular body at the aperture. Prior to deployment of the exclusion device, an extension system, useful for extending a branch vessel flow element of the exclusion device, is first deployed intravascularly, from an incision through the skin of the patient and into one of the femoral arteries, into the branch vessel at the aneurysmal location, and thence guided further along the branch vessel until a location is reached where it may be retrieved from the branch vessel through an incision through the skin and into the branch vessel at a location where the branch vessel may be so accessed. The extension system is typically routed along a guide wire to deploy an extension system through each branch lumen which would otherwise be excluded by the exclusion device. The first end of each such extension system is attached to the branch vessel flow element integral with the main body of the tubular device which is held in a deployment sheath in a deployment catheter prior to delivery thereof to the aneurysmal site, and the second end of the expansion system once guided by the guide wire, is held at a remote location exterior to the patient's body, while extending from the deployment catheter through the aneurysmal location and the branch vessel to the location exterior of the patients skin.

In one aspect, the extension system(s) is composed of string like leads, which are secured to the branch vessel flow elements such that, upon deployment of the exclusion device to the aneurysmal site, the pulling of the leads from the remote location adjacent to the patient's skin causes the branch vessel flow element to be extended outwardly from the main body of the exclusion device at a location aligned with the location of the patient's branch vessel. Once the leads are properly positioned, the tubular main body of the exclusion device is then intravascular routed to an aneurysmal flow lumen location while the leads of the extension system are correspondingly pulled outward from the patient's body at remote locations, so as to maintain a non-overlapping physical configuration between the extension system and the exclusion device as it is guided to the aneurysmal location. When the exclusion device reaches the aneurysmal location, the sheath holding the main body portion is exposed and/or slowly withdrawn as the practitioner deploying the main body portion aligns the main body portion so as to span the aneurysmal location and to properly position the apertures and branch vessel flow elements to be positioned in alignment with the location of the branch vessels on the vessel wall. As the main body is deployed, the extension system(s) is used to pull the ends of the branch vessel flow elements on the main body into a position extending outward from or in the direction of the branch vessel location(s). The extension system may then be removed or may be later removed after deployment of the secondary extension member (anchoring element, fixation member, or stent) which is likewise routed to the aneurysmal location into the interior of the main body of the exclusion device to the interior of the branch vessel flow element extending into or in the direction of the patient's branch vessel wherein the secondary extension member is deployed within the branch vessel flow element and expands against and seals against the branch vessel flow element and the wall of the branch vessel.

In one aspect, the extension system(s) includes at least two leads and the flange includes at least two apertures therein for each lead, adjacent to the end of the branch vessel flow elements extending from the main body of the exclusion device. Prior to deployment in the deployment sheath and catheter delivery system, each lead is marked to identify it from the adjacent lead. Additionally, a balloon or other inflation device may be deployed within the circumference of the aperture from which the branch vessel flow element extends on the main body. When deployed, the leads are pulled from a position exterior to the patient's body to extend the distal end of the branch vessel flow element from the main body of the exclusion device, and the balloon may be inflated to open the main body into an open tubular shape. The secondary extension member may then be deployed, and the leads are removed by simply pulling on one of the opposed ends of each lead until the other end of the lead is pulled from the patient's body at the remote location. For each branch vessel spanned by the exclusion device, one branch vessel flow element and one secondary extension member are used to ensure blood flow from the tubular interior of the main body and into the branch vessel, while sealing the aperture and branch lumen wall to prevent blood flow to the aneurysmal site.

### BRIEF DESCRIPTION OF THE DRAWINGS

Embodiments according to the invention are described in the present specification and illustrated in the appended drawings. It is to be noted, however, that the specification and appended drawings illustrate only certain embodiments and are, therefore, not to be considered to be limiting in scope.

FIG. 1 is an artists rendering of a sectional view of an aneurysmal aortic arch, wherein the aortic arch is dilated in the vessel region opposite the branch vessels emanating therefrom;

FIG. 2 is a perspective view of an exclusion device useful for excluding fresh blood to the aneurysmal region of the aneurysmal aortic arch of FIG. 1;

FIG. 3 is a view of the artists rendering of a sectional view of an aneurysmal aortic arch of FIG. 1 showing the exclusion device of FIG. 2 deployed therein;

FIG. 3A is a perspective view of a branch vessel flow element constructed of a PTFE tube whose end is formed in a petal arrangement for engagement with a silicone annular ring which will act as a flange at the end of the PTFE tube;

FIG. 3B is a cross sectional side view of the flange of FIG. 3A taken at 3B-3B;

FIG. 3C is an end view of the tube of FIG. 3A with the petals of tube in their pre-engagement configuration with the silicone flange;

FIG. 3D shows an end view similar to that shown in 3C, with the interengagement/overmolding of the tube petals and flange completed;

FIG. 3E shows a perspective view of the combination tube-flange of FIG. 3A in a pre-main body attachment configuration;

FIGS. 4 and 4A are partial sectional views of the deployed exclusion device of FIG. 3 at one of the branch aperture locations, showing the extension of a synthetic secondary flow lumen from the main body of the exclusion device and into the patient's branch vessel;

FIG. 5 is a perspective view of the thin walled branch vessel flow element portion of the synthetic secondary flow lumen of FIG. 4;

FIG. 6 is a perspective view of the main body of the exclusion device of FIG. 2, prior to deployment in the aneurysmal aortic arch;

FIG. 7 is a partial cutaway view of the exclusion device of FIG. 6, showing the positioning of an inflation device within the envelope of the one of the thin walled branch vessel flow elements thereof;

FIG. 8 is a view of the main body of the exclusion device of FIG. 6, in a collapsed state for placement in a delivery sheath;

FIG. 9 is a view of a delivery sheath having the main body of the exclusion device of FIG. 6, showing the leads and guide wire used in the placement of the exclusion device extending therefrom;

FIG. 10 is an artists rendering of the aneurysmal aortic arch of FIG. 1 showing guide wire and one set of leads extending into a branch vessel;

FIG. 11 is an artists rendering of the aneurysmal aortic arch showing the leads extended into each of the branch vessel and the delivery device partially extended through the arch;

FIG. 12 is an artists rendering of the aneurysmal aortic arch, showing the delivery device ready to deploy the main body of the exclusion device;

FIG. 13 is an artists rendering of the aneurysmal aortic arch, showing the main body of the exclusion device partially deployed from the delivery device;

FIG. 14 is an artists rendering of the aneurysmal aortic arch, showing the main body of the exclusion device partially deployed from the delivery device, and one of the thin walled branch vessel flow elements positioned in its extended position;

FIG. 15 is an artists rendering of the aneurysmal aortic arch, showing the main body of the exclusion device fully deployed from the delivery device, and each of the thin walled branch vessel flow elements positioned in its extended position; and

FIG. 16 is a partial cutaway view of the main body of the exclusion device positioned in the aneurysmal aortic arch as in FIG. 15, showing the initiation of the deployment of a synthetic secondary extension in the thin walled branch vessel flow element.

### DETAILED DESCRIPTION

Reference now will be made to details of exemplary embodiments according to the invention. While the invention will be described in conjunction with these embodiments, it is to be understood that the described embodiments are not intended to limit the invention solely and specifically to only these embodiments.

Methods and apparatus for stabilizing and treating an aneurysm include deploying an exclusion device, such as a stent graft, in the aneurysmal flow lumen to span the aneurysmal location therein and to seal the aneurysmal location off from further blood flow thereto. In the case of a thoracic aneurysm of the aortic arch, methods and apparatus for the treatment thereof include positioning an endovascular stent graft in the aneurysmal site in the aortic arch, wherein the stent graft includes at least one, and in the embodiment described herein, three, apertures therein, and at least one of these apertures includes an branch vessel flow element therein for sealing engagement with the branch lumen, having a generally tubular profile enabling blood flow therethrough. In one aspect, the branch vessel flow element may include a flange sewn into or otherwise attached to the tubular graft material and a branch vessel flow element extending from the aperture and into a branch artery located in the thoracic aortic arch. Each of the apertures is alignable with the branch vessel flow element extendable into or in the direction of, the branch artery, and are further secured for sealing engagement with the branch lumen by separate deployment of a secondary extension member (stent) which sealingly engages both the branch vessel flow element and the branch artery wall to accommodate blood flow into the branch artery from the interior volume of the stent graft and to simultaneously span any gap between the outer surface of the main body of the stent graft and the aneurysmal artery wall. The stent graft excludes the weakened vessel wall at the aneurysmal site from further exposure to blood flowing through the aorta, but, as a result of the branch configuration, allows blood flow from the tubular body of the stent graft to the branch arteries, even where the stent graft extends over the exit of the brachiocephalic trunk, the left common carotid artery and the left subclavian artery from the aorta. Moreover, the stent graft may thus have a length such that it extends over the exit of the branch arteries from the aorta to thereby enabling sealing of the stent graft against the aorta wall at a healthy blood vessel wall location and thereby enable sealing off of the aneurysmal site from further blood flow thereto. Furthermore, by separately providing the branch vessel flow element, as an integral portion of the main body of the stent graft, and the secondary extension member in a secondary or later procedure, the bulk of the exclusion device is reduced, and thus the crossing profile of the delivery vehicle may be reduced.

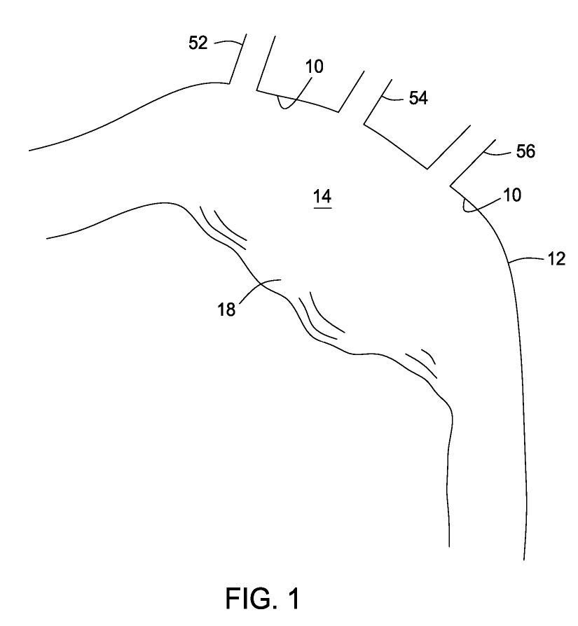

Referring initially to FIG. 1, there is shown an artists rendering of an aneurysm of the thoracic aortic arch 12, such that the arch 12 is enlarged at an aneurysmal site 14 wherein the aorta wall 10 is distended and stretched. The aneurysmal site 14 forms an aneurysmal bulge or sac 18. If left untreated, the aneurysmal sac 18 may continue to deteriorate, weaken, increase in size, and eventually tear or burst. The arch 12 generally extends upwardly and laterally from the heart (not shown), such that at the aortic arch three branching arteries, the brachiocephalic trunk (52), the left common carotid artery (54) and the left subclavian artery (56) extend from the thoracic aorta 12. The aneurysm in this FIG. 1 implicates the aorta wall 10 in the region of the branch arteries, as the aortic arch 12 is dilated at the branch artery 52, 54 and 56 locations.

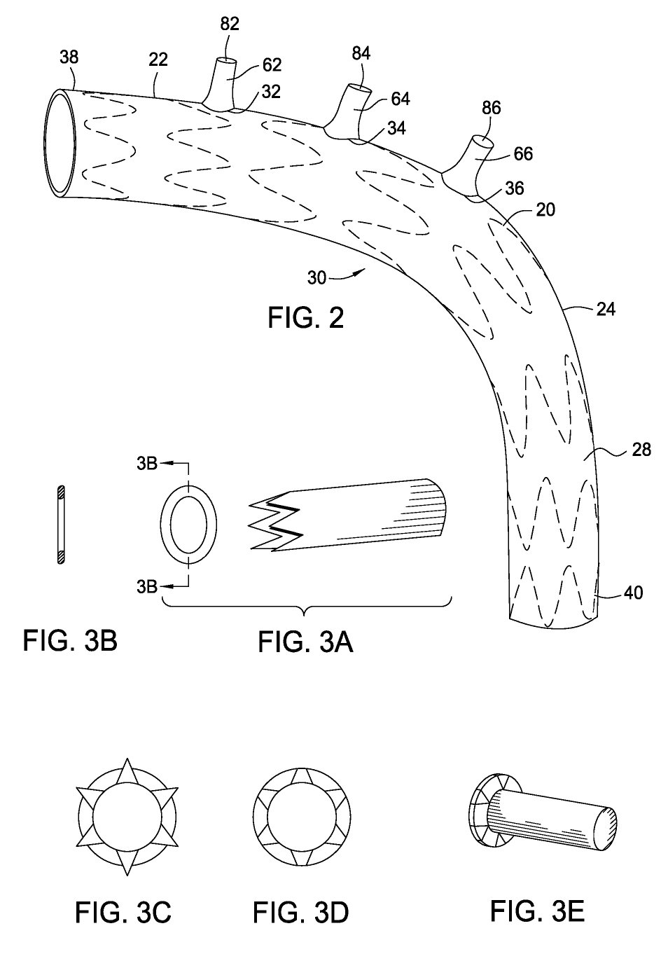

FIG. 2 is an exterior side view perspective of a stent graft 30 according to the one embodiment of the present invention. There is shown generally a stent graft 30 comprising a stent framework 20 (Shown with dashed lines in FIG. 2) and graft material 22 exterior to (though it could be internal to) the stent framework 20, which together form an integral tubular structure which provides a main body 24 which is self supporting in a open tubular shape when deployed in an aneurysmal aortic arch, and also provides a sealing force, at the opposed ends 38, 40 of the stent graft 30, to sealingly engage against healthy tissue of the aorta wall 10 located to either side of the aneurysmal site 14. In the embodiment shown of FIG. 2, there are three openings seen at 32, 34 and 36 extending through the wall of the main body 24 of the stent graft 30, and from each of which a branch vessel flow element 62, 64 and 66 respectively extends for extension into, or in the direction of, a branch artery once the stent graft is deployed. Opening 32 and branch vessel flow element 62 accommodate the exit of the brachiocephalic 52 trunk from the aortic arch 50, opening 34 and branch vessel flow element 64 accommodate the exit of the left common carotid artery 52 from the aortic arch 50 and opening 36 and branch vessel flow element 66 accommodate the exit of the left subclavian artery 56 from the aortic arch 50. The construction of the integral branch vessel flow elements from the same woven polyester (Dacron) fabric as the main graft material, with the branch vessel flow element sewn to the main body, provides a bulky arrangement of the device when compressed for insertion into or containment by a delivery catheter.

In an alternate embodiment of the branch vessel flow elements, the element is constructed of a very thin, durable material such as PTFE. Since the connection of PTFE to polyester directly has not proven successful in the past, it was necessary to investigate other methods or configurations. One configuration which has proven successful is to mate a PTFE tube to a silicone flange by overmolding the PTFE material on the flange and then sewing the flange of the now combined tube/flange structure to the polyester material of the main body.

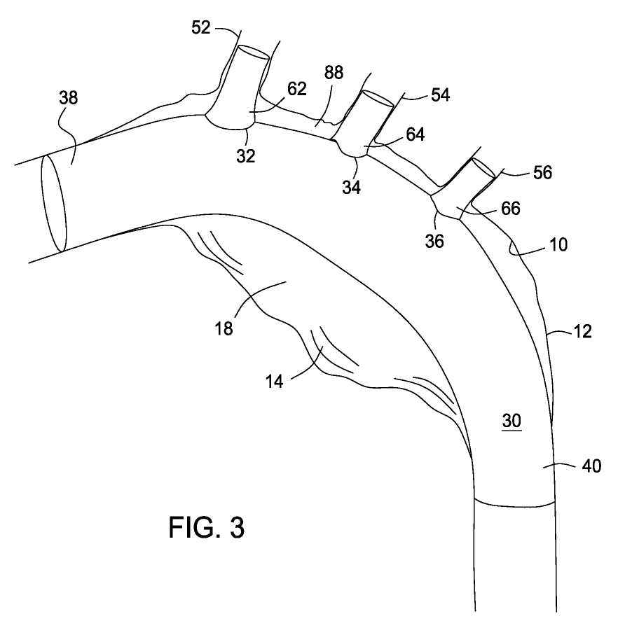

FIG. 3A shows a PTFE tube whose left end has been configured into a circularly oriented set of petals. The ends of the petals are configured to pass through the annular flexible silicone flange. A cross section of the silicone flange is shown in FIG. 3B. FIG. 3C shows the PTFE tube positioned inside of the silicone flange with the petals of the PTFE tube extended laterally outward. FIG. 3D shows the petals wrapped around and over molded on the silicone flange. FIG. 3E shows a perspective view of the PTFE tube attached to the silicone flange which can then act as an anchor or intermediate member to connect and seal the PTFE tube acting as a branch to the main body, by being sewn, glued, or otherwise bonded in a seal promoting manner to the main body.

In one embodiment, the flanges can be an elastomer, such as biocompatible silicone.

Once deployed, the stent graft 30 is intended to provide a synthetic flow conduit across or past the aneurysmal sac 18 of the aortic arch 12 and to seal off the aneurysmal sac 18 from further blood flow. The stent graft 30 is sized so that, upon deployment thereof in the aortic arch 12, as shown in FIG. 3, the diameter of the stent graft 30 is slightly larger than the normal, healthy diameter of the aortic arch 12 where the graft 28 material adjacent to the ends 38, 40 of the stent graft 30 contacts the wall 10 of the aortic arch 12, and it has a length sufficient to span the aneurysmal sac 18 of the aorta 12 and sealingly contact the aorta 12 at healthy tissue regions of the blood vessel wall 10 upstream and downstream of the aneurysmal sac 18. Such sealing may, but need not, include the region of the wall 10 between the branch arteries 52, 54 and 56, as well as regions distal, i.e., to the heart side of the aorta 10, from the aneurysmal sac 18. Additionally, the plurality of openings 32, 34 and 36 are, when the stent graft 30 is properly deployed, positioned adjacent to respective openings of the branch arteries into the aorta 12, such that the branch vessel flow elements 62, 64 and 66 emanate from openings 32,34 and 36, and extend from apertures in the stent graft 30 to a sealed engagement location within the interior wall of the respective branch artery 52, 54 and 56.

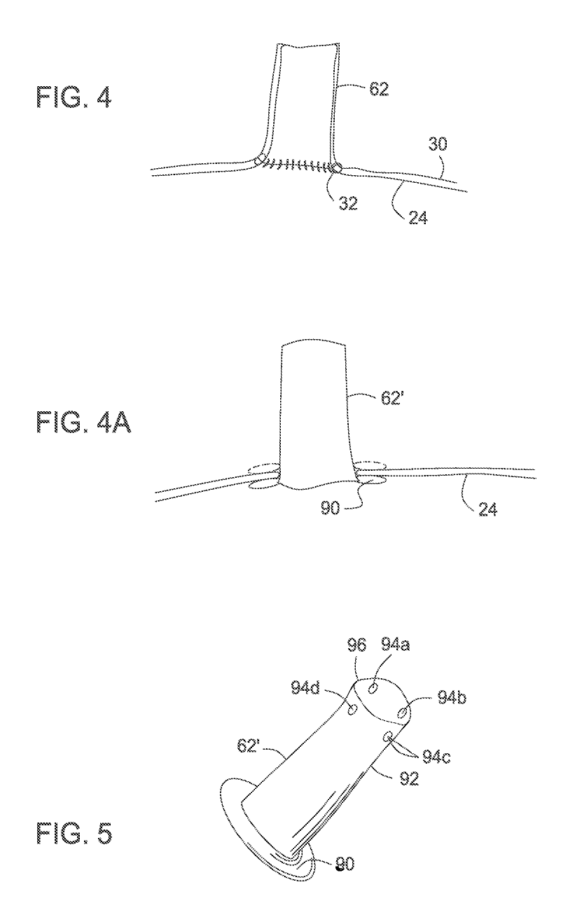

Referring now to FIG. 4, the specific construction of one embodiment of the branch vessel flow elements 62, 64 and 66 are shown (branch vessel flow element 62 being representative of all three and thus only branch vessel flow element 62 is shown), where branch vessel flow element 62 and its attachment with the aperture 32 of the main body 24 of stent graft 30 is shown as an exemplar, it being understood that in this embodiment, each branch vessel flow element 62, 64 and 66 and its attachment thereof to the main body 24 of the stent graft, is substantially similar. The branch vessel flow elements are sewn using attachment sutures along a circumferential seam (not shown) to the main body.

Another embodiment of the branch vessel flow element as discussed for FIGS. 3A-3E above and as shown in FIG. 4A, branch vessel flow element 62 includes a flange 90 shown in cross section and with the branch vessel flow element 62′ extending from aperture 32 in main body 24 of stent graft 24. Flange 90 is secured to the graft material 30 surrounding the aperture 32 such as by sewing the abutment flange 90 thereto.

Referring to FIG. 5, a branch graft combination is shown such that lead apertures 94a-d are seen adjacent to the distal end 96 of branch vessel flow element 62′, such ends being disposed opposite abutment flange 90. During deployment, leads (not shown) extend through the apertures 94a-d and along the branch artery therefrom, such that the leads may be used to pull the distal end 96 of the branch vessel flow element 62, 62′ away from the main body 24 of the stent graft 30.

In the embodiment described herein, the stent graft 30 is deployed into an thoracic aortic arch 12 adjacent to and spanning branch vessels therefrom, to provide an exclusion of the blood flow to the aneurysmal sac 18 and simultaneously enabling secure blood flow into the branch arteries 52, 54 and 56. Generally, an assembly of the stent graft 30 having the branch vessel flow elements 62, 64 and 66 disposed thereon and positioned within the vascular lumens then have stents each separately introduced into the aorta intravascularly by use of delivery sheaths and/or catheters to secure the branch vessel flow element in the vascular lumens. A similar arrangement could be used for providing a branch connection to the renal arteries when spanning an abdominal aortic aneurysm (AAA) in the descending aorta.

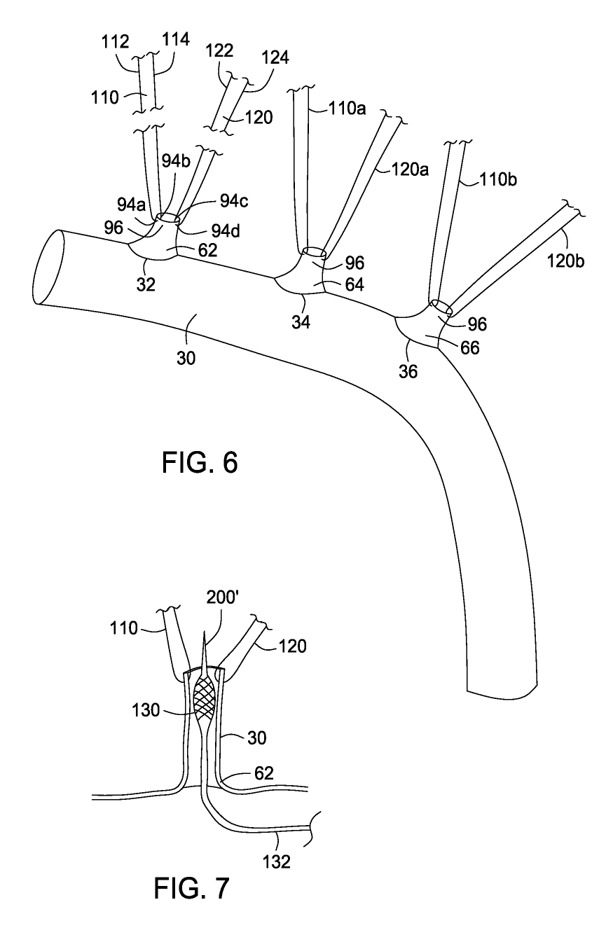

The stent graft 30 of the present invention is intended to be deployed endovascular, i.e., by being routed to the aneurysmal site 14 in a delivery vehicle through a patients' vascular system, and then released or deployed from the delivery vehicle to span and sealingly exclude the aneurysmal sac 18 from further blood flow. However, in this embodiment, unlike a self expanding stent graft, the branch vessel flow elements 62, 64 and 66 will not self deploy, i.e., they will not regain a desired tubular shape without further intervention. Therefore, referring now to FIG. 6, the stent graft 30 is shown prior to the deployment thereof. Specifically, each of the branch vessel flow elements 62, 64 and 66 is shown positioned within its respective apertures 32, 34 and 36, such that the distal end 96 of each is spaced from the adjacent apertures 32, 34 or 36. Referring initially to branch vessel flow element 62, which will be described as typical of the structure and arrangement of all three branch vessel flow elements 62, 64 and 66 of this embodiment, there are provided the lead apertures 94 a-d as previously described with respect to FIG. 5, and additionally, two thread-like leads 110, 120 are provided, such that the lead 110 extends through circumferentially adjacent to lead apertures 94a, 94b, and lead 120 extends through circumferentially adjacent lead to apertures 94c, 94d. The leads 110, 120 are then positioned, such that the opposed ends 112, 114 of lead 110, and the opposed ends 122, 124 of lead 120, are drawn together. The length of each lead 110, 120 is sufficient to extend from a delivery sheath (shown in FIG. 9) which will be located, when the stent graft 30 is to be deployed, adjacent to an incision of into an iliac artery near the groin, through the iliac artery and through the aortic arch 12, and thence into one of the branch arteries to a position where it exits the body at an incision in the upper body (chest or neck) where the branch artery is relatively close to the skin. Leads 110 and 120 are coupled to branch vessel flow element 62 as described, leads 110a and 120a are similarly coupled to branch vessel flow element 64 and leads 110b and 120b are likewise coupled to branch vessel flow element 66.

To deploy the stent graft 30, the stent graft 30 main body 24 with the branch vessel flow elements 62, 64 and 66, attached thereto, the main body 24 is collapsed or compressed to fit into a sheath of an intravascularly deployable catheter. Prior to deployment of the stent graft, the leads 110, 120 are deployed, as previously described, through apertures 94a-d in each of branch vessel flow elements 62, 64 and 66.

Referring now to FIG. 7, the stent graft 30 is shown in partial cutaway, such that the deployment of a stent mounted on a balloon 130 at the end of a catheter 132 is shown. Each branch vessel flow element 62, 64 and 66 is provided with a separate stent mounted balloon, only stent mounted balloon 130 is shown. A catheter guide wire 200′ is extended through the length of the tubular main body portion 24 guides the stent mounted balloons into place.

Once leads 110, 120 are attached to their respective branch vessel flow element, the stent graft 30 and the main body 24 may be compressed for loading in a delivery sheath or delivery shroud.

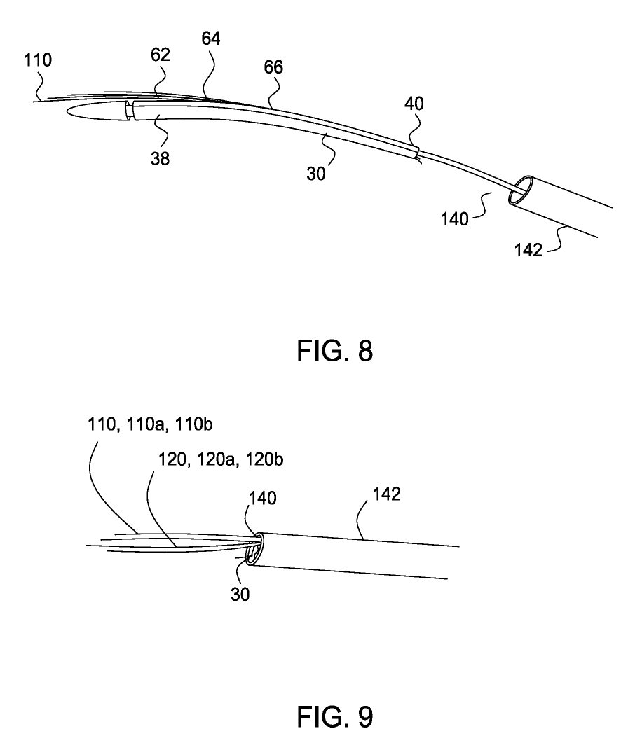

In this embodiment, the stents 20 are preferably manufactured from a shape memory material, such as nitinol, such that the stents 20 will attempt to regain their original shape upon being released from restraint at body temperature. In this embodiment, the stents 20 are preferably hoops each wound in a zigzag configuration from a single length of shape memory materials such as nitinol. Alternatively, a non-shape memory material may be used to configure the stents, and such stents may need to be expanded, in situ, with a balloon provided within the stent graft for this purpose. With the stent graft 30 sufficiently compressed around a central catheter member, it is moved relative to the open end 140 of delivery sheath 142 shown in FIG. 8, such that the stent graft 30 is fully contained within open end 140 of delivery sheath 142, and leads 110, 120 extend from the open end 140 as shown in FIG. 9. Alternatively, a releasable stitched or splitable sheath (or shroud) can be utilized where leads (e.g., 110, 120) connected to the ends of the branch vessel flow elements are threaded through openings in the side of the sheath. The locations of the threadable openings in the releasable sheath are spaced and located approximately opposite the vascular lumens into which the branch vessel flow elements are to extend upon deployment of the stent graft. In this way, the leads can pull the branch vessel flow elements into the vessel lumens without having to pull the branch vessel flow elements along the stent graft after it is deployed. The catheter central member shown in FIG. 8 extends down the entire length of delivery sheath 142. Additionally, leads 110, 110a, 110b, 120, 120a and 120b are coded to enable the surgeon or practitioner to determine which set of leads corresponds to specific ones of branch vessel flow elements 62, 64 and 66.

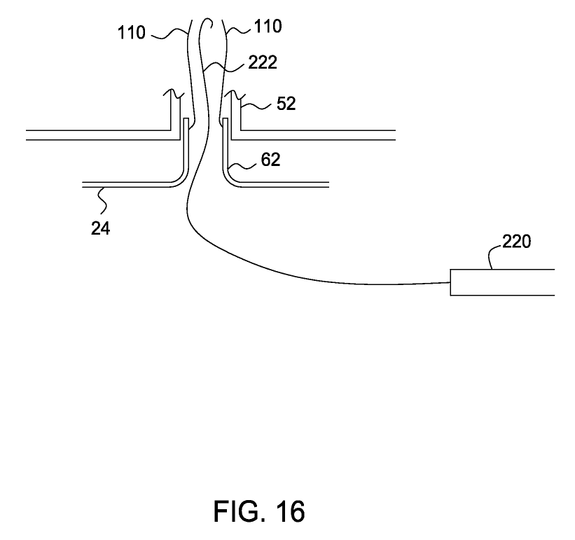

In addition to the main body 24 of stent graft 30, the branch stabilizing/securing/anchoring stents (self-expanding or balloon expandable) need subsequently to be deployed with delivery catheters. As with the stent graft 30 main body 24, each of the anchoring stents is inserted into the respective branch where it will be deployed over a guidewire that has previously been placed into the branch (shown in FIG. 16).

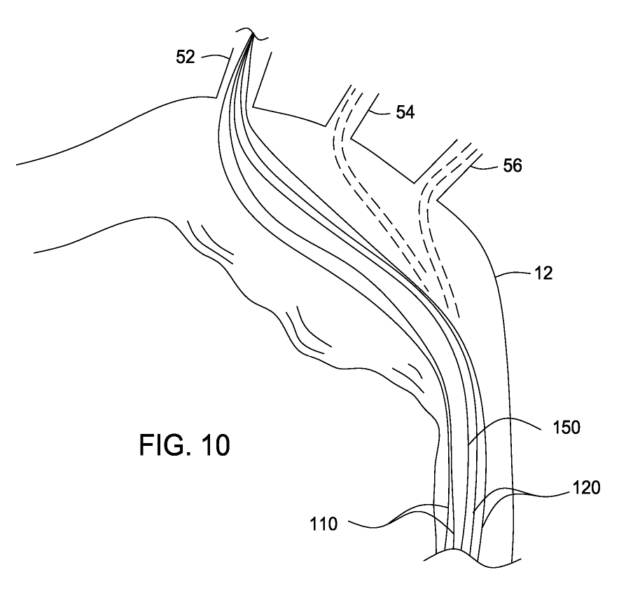

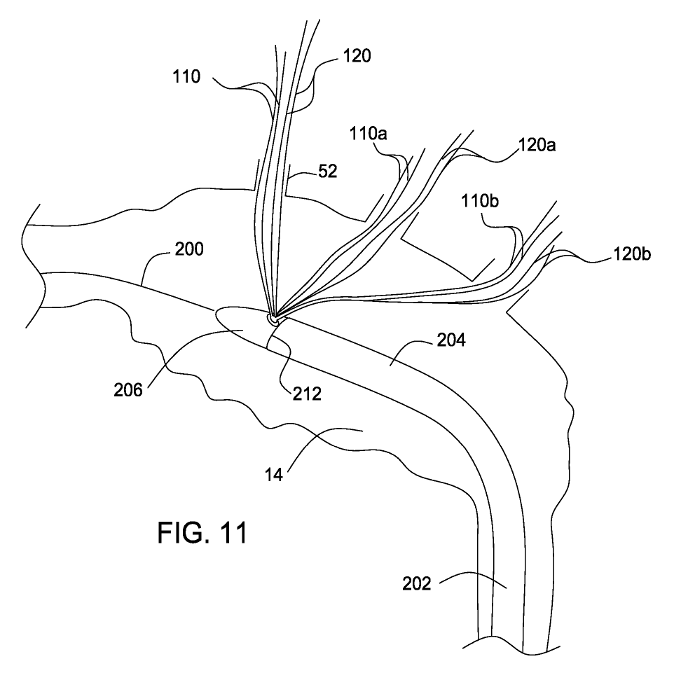

Referring now to FIG. 10, the initiation of the deployment of the stent graft 30 is shown. Initially, a guide wire 150 having leads 110, 120 connected thereto is directed, from an incision into an iliac artery adjacent to the patients' groin, and guided through the arch and into the brachiocephalic artery 52. Similar guide wires (shown approximated by dashed lines) are provided to extend the leads 110a, 120a into left common carotid artery 54, and leads 110b, and 120b into left subclavian artery 56. In each case, the guide wire is guided up the branch artery 52 (or 54, 56) to a location where the branch artery is accessible through the skin, where an incision through the skin, and into the branch artery was previously performed, and the leads 110, 120 (or 110a, 120a and 11b, 120b) are recovered. The guide wires are then removed.

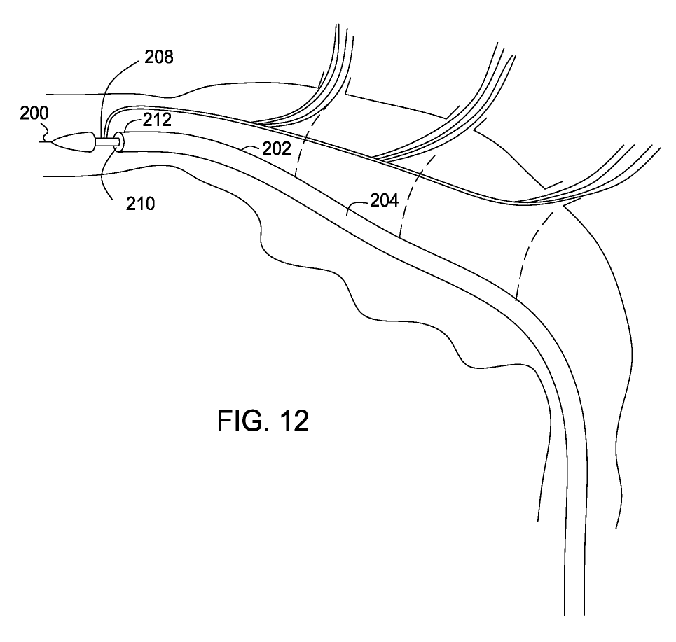

After the leads 110, 110a, 110b and 120, 120a, and 120b are properly deployed and secured external to the body at a remote site (not shown), a delivery catheter guide wire 200 may be inserted into a groin incision and the femoral artery (not shown), and guided to a position upstream of, in a blood flow sense, the aneurysmal site 14 of the arch. As shown in FIG. 11, delivery catheter 202 includes an outer tubular housing (sheath) 204, having a generally hollow tubular interior 210 (FIG. 12). A tapered introduction portion 206 is fixed to the catheter central member 208 (FIG. 12). To deploy the main body, the catheter 202 is guided up the guidewire 200 to a position upstream of the aneurysmal site 14. The leads 110, 110a, 110b and 120, 120a and 120b extend from the delivery sheath 142 of the catheter 202 such that they exit the openable end 112 and do not extend through the tapered introduction portion 206. This may be accomplished by providing a slot inwardly of the openable end 112 to accommodate the leads as necessary. The catheter 202 is then moved along the guidewire 200, until as shown in FIG. 12, the end 212 thereof is disposed at the upstream deployment location of the main body 24 of the stent graft 30. As the catheter 202 is guided into the positions shown in FIGS. 11 and 12, the leads 110, 110a 110b and 120, 120a and 120b are pulled from their respective remote locations at the same rate that the catheter 202 is moving into the body, to ensure that the catheter 202 does not pass by, and possibly bind, any one of the leads. The delivery sheath 142 is likewise positioned within the catheter, such that its open end 140 is aligned with the openable end 212 of the catheter 202.

In an alternate configuration, the catheter sheath can be a seamed splitable type with a pull wire or pull chord. The leads can be routed out holes, openings or splits in the side of the sheath (e.g., as shown in dashed lines) so that the need for manipulation of the leads is minimized.

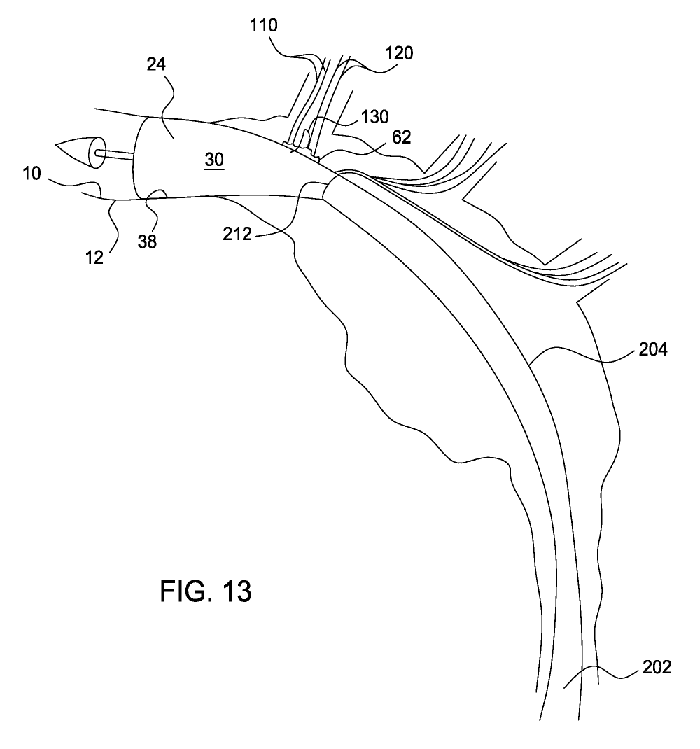

In one embodiment, to deploy the main body 24, the catheter 202 housing 204 after having been rotationally aligned so that branch openings face branch vessels as precisely as possible, the catheter 202 housing is retracted as shown in FIG. 13, exposing the stent graft 30 and a push rod or stop (not shown) is held firmly against the end 40 (not shown in this Figure) of the compressed stent graft main body held within the delivery sheath 204. Then, the delivery sheath 204 is withdrawn incrementally, so that the stent graft 30 main body 24 begins to deploy from the sheath 204 as shown in FIG. 13. As the stent graft end 38 expands under the bias of the shape memory stents, the end 38 will engage against the inner wall 10 of the aortic arch 12, to enable sealing of the stent graft 30 therewith. Likewise, the first of the branch vessel flow elements, branch vessel flow element 62, is exposed. Prior to engagement of the end 38 of the main body 24 with the aorta 12 wall 10, the sheath 204 may be both rotated, in situ or pulled back and rotated and then re-positioned to ensure alignment of the flanges with the branch arteries 52, 54 and 56, as well as to ensure placement of the portion of the stent graft 30 interior of end 38 over a sufficient length of healthy aorta wall 10 to ensure sealing of the stent graft 30 to the wall 10. To enable proper positioning of the main body 24, the main body 24 of the stent graft 30 includes therewith radiological markers, such that the practitioner may visualize fluoroscopically the location of the stent graft vis-à-vis the branch vessels 32, 34 and 36, as well as its rotational alignment in the aneurysmal aorta.

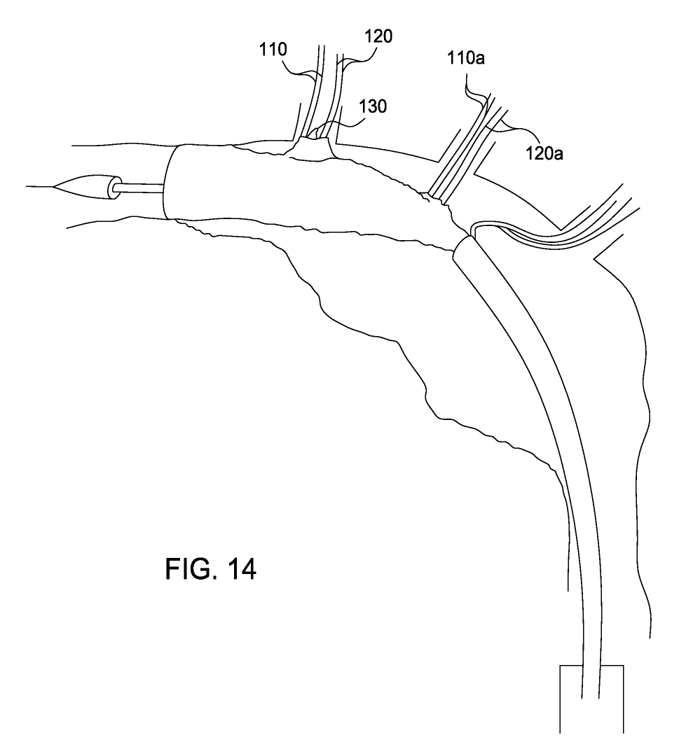

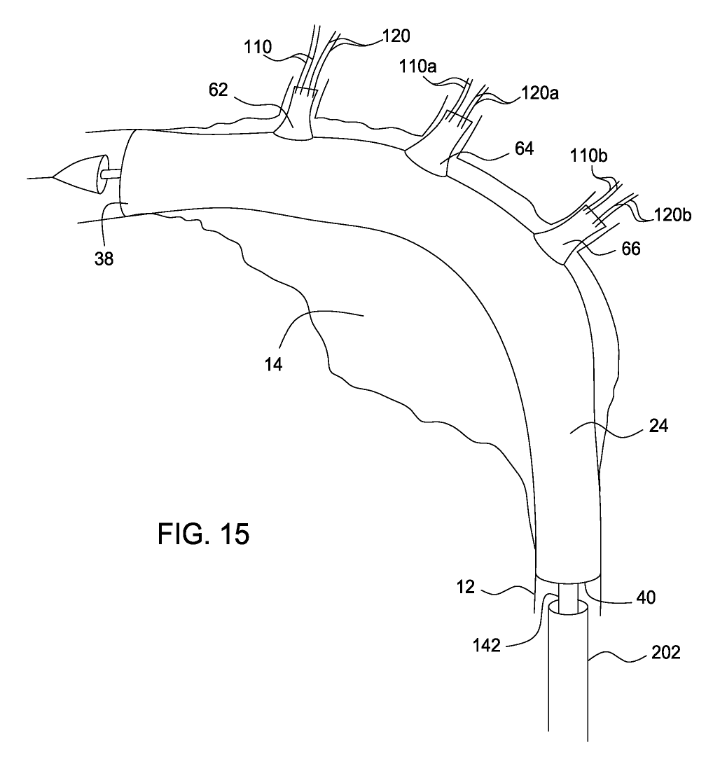

As the sheath 204 is pulled to the position shown in FIG. 13, the ends of the leads 110, 120 are pulled, to extend the branch vessel flow element into the extended position shown in FIG. 14. The leads are preferably held in this position, while the balloon 130 is inflated, to open branch vessel flow element 62 into a generally circular tubular profile. This is repeated with branch vessel flow elements 64 and 66 as the delivery sheath is retracted, until the main body 24 of the stent graft 30 is deployed as shown in FIG. 15. Thence, the catheter 202 may be withdrawn through the incision and branch anchoring stent delivery catheters may be introduced and deployed in each branch artery. To deploy an anchoring stent into branch vessel flow element 62, a stent anchoring catheter guidewire 222 is guided up the aorta and then guided into branch artery 52 as shown in FIG. 16 which is a partial cutaway view of the aneurysmal site showing the detail of the branch vessel flow element 62 adjacent to branch artery 52. Once the stent anchoring catheter guidewire 222 is deployed inwardly of branch artery 52, the stent anchoring catheter 220 can be directed therein as shown in FIG. 16. Thence, a push rod or stop (not shown) within the secondary extension sheath 220 is held stationary as the stent sheath is retracted, deploying the stent or alternately, when a balloon expandable stent is used, the balloon upon which the stent is carried to the site is inflated using a contrast saline solution. Then, one end of each of leads 110, 120 is pulled, such that the leads 110, 120 are pulled trough the lead apertures 94a-d and thence from the body. This procedure is repeated to deploy a stent into branch vessel flow element 64 and a stent into branch vessel flow element 66. Once the anchoring stents are deployed and the leads removed, the stent graft is secured to prevent blood flow to the aneurysmal site 14 as shown in FIG. 3. The incisions in the patient are then closed.

Thus, there is shown and described an exclusion device useful for the exclusion of a diseased or damaged condition of a flow lumen, such as an aneurysmal condition in an aorta, where branch lumens extend off of the flow lumen adjacent to, or within, the aneurysmal position of the aorta. The stent graft 30 as shown includes a main tubular body, having the capability, when deployed, to span the aneurysmal site or sac and seal against healthy wall tissue at opposed end of its tubular main body. Additionally, the main body includes apertures, extending through the wall thereof, to enable fluid flow from within the main body to occur through the aperture. These apertures are alignable with branch vessels from the aorta or other flow lumen, and include a branch vessel flow element deployable with the exclusion device to extend in, or in the direction of, the branch lumen and an anchoring stent to ensure sealed, flow enabling, engagement between the apertures and the branch lumen.

The configuration of the stent graft 30 and the separable deployment of anchoring stents with the branch arteries allows use of sheaths and catheters with smaller crossing profiles, as the maximum bulk of the stent graft at the branch vessel is significantly reduced by using a thin walled elastomer flange with a thin membrane tubular branch such as PTFE connected to the silicone flange, with no inherent structural rigidity. Thus, a stent graft incorporating the features described with respect to stent graft 30 may be delivered more readily through restricted areas of body flow lumens, enabling greater success at deployment of stent grafts into patients.

The materials making up the stent portion of the present invention may be a metal. Metal stents are known in the art, and metals such as stainless steel and nitinol (NiTi) have been used, although the shape memory material nitinol is preferred. In addition, various iron alloys have been used such as iron platinum, iron palladium, iron nickel cobalt titanium, iron nickel carbon, iron manganese silicon and iron manganese silicon chromium nickel. Alternatively, the stents may comprise one or more biocompatible polymeric materials, preferably, non-degradable polymeric materials. Generally the diameter of the metal or polymeric wire used for construction of the stent is between 0.005 inches to 0.02 inches.

The graft material may be any known in the art, and generally is a material, for example, that can bend and reshape as the stent is expanded within the vessel. For a self-expanding stent graft, the graft material can be expanded with the expansion force inherent in the stent; alternatively, the stent graft can be expanded with a balloon catheter. Once a stent graft is expanded in the vessel, the stent graft will retain its expanded properties. In addition to its elastic nature, the graft material also is of a nature that, after expansion, it exhibits low residual stress to prevent wear and tear. Control of the elasticity of the graft material can control the necessary inflation pressure of the covered stent.

The thickness of the graft material optionally is minimized to reduce the overall cross sectional thickness of the stent graft and the pressure necessary to deploy it. Generally, the graft material will be thinner than 0.005 inch, and may be thinner than 0.002 inch. The thickness of the graft material is generally consistent over the length of the stent.

In the embodiment of the stent graft shown in FIG. 3, the ends of the graft material extend beyond the marginal edges of the stent graft. This arrangement is one of various arrangements of the position of the graft material with respect to the stent, as in other embodiments according to the present invention the ends of the graft material are coincident with the ends of the stent. The ends of the graft portion of the stent graft are configured to prevent fraying, which may be accomplished by heat fusion, binding, or by folding the end of the graft material back and sewing it to the wall 24. Also, the graft material may be located on the interior of the stent framework, on the exterior of the stent framework, or the graft material may be located in the interstitial spaces between the portions or sections of the stent framework as shown in the Figures herein. The graft material may include more than one layer or plies. The graft material preferably is non-porous to prevent the entry of inflammatory agents and/or embolic debris into the lumen of the stent graft. In some embodiments, the graft material may include a coating of non-porous material over one or more porous layers. The embodiments of the stent grafts shown herein show a continuous cylindrical wall, but for the locations of the openings 36, 34 and 32.

The graft material may be any biocompatible material that demonstrates sufficient elasticity, is mechanically stable in vivo and allows attachment of the flanges. In a preferred embodiment, a non-resorbable polymer is used to provide such necessary characteristics. Representative examples of non-degradable polymers include poly(ethylene-vinyl acetate) (“EVA”) copolymers, silicone rubber, polyamides (nylon 6,6), polyurethane, poly(ester urethanes), poly(ether urethanes), poly(ester-urea), polypropylene, polyethylene, polycarbonate, polytetrafluoroethylene, expanded polytetrafluoroethylene, polyethylene teraphthalate (Dacron), polypropylene or their copolymers. In general, see U.S. Pat. Nos. 6,514,515 to Williams; 6,506,410 to Park, et al.; 6,531,154 to Mathiowitz, et al.; 6,344,035 to Chudzik, et al.; 6,376,742 to Zdrahala, et al.; and Griffith, L. A., Ann. N.Y. Acad. of Sciences, 961:83-95 (2002); and Chaikof, et al, Ann. N.Y. Acad. of Sciences, 961:96-105 (2002). Additionally, the polymers as described herein also can be blended or copolymerized in various compositions as required.

While the present invention has been described with reference to specific embodiments, it should be understood by those skilled in the art that various changes may be made and equivalents may be substituted without departing from the true spirit and scope of the invention. In addition, many modifications may be made to adapt a particular situation, material or process to the objective, spirit and scope of the present invention.

All references cited herein are to aid in the understanding of the invention, and are incorporated in their entireties for all purposes.

1. An intravascular treatment device deployable in conjunction with a deployment member for excluding an aneurysmal site in a body therefrom, comprising:
a tubular exclusion portion constructed of a woven or braided textile material positionable to span the aneurysmal site and including at least one aperture therewith positionable adjacent to a branch flow lumen;
a branch vessel flow element constructed of a thin walled deformable PTFE tube, where a proximal portion of said tube material is fluid tightly fixed to a flexible material circumferential flange;
wherein said circumferential flange is substantially fluid tightly fixed to said tubular exclusion portion around said at least one aperture so as to form a substantially fluid tightly sealed branch flow lumen from said tubular exclusion portion to and through said branch vessel flow element.

2. The intravascular treatment device of claim 1, wherein said flexible material of said circumferential flange includes silicone.

3. The intravascular treatment device of claim 1, wherein said thin walled deformable tube is overmolded on said circumferential flange.

4. The intravascular treatment device of claim 3, wherein said thin walled deformable tube includes a first end, said first end configured to include a plurality of petal like extensions which are engageable and moldable to said circumferential flange.

5. The intravascular treatment device of claim 1, wherein said tubular exclusion portion includes Dacron.

6. The intravascular treatment device of claim 1, wherein said thin walled deformable tube is comprised of e-PTFE.

7. The intravascular treatment device of claim 1, wherein said thin walled deformable tube has a wall thickness of less than 0.002 inches.

8. The intravascular treatment device of claim 1, wherein said thin walled deformable tube includes a first end portion adjacent to said aperture and a distal end portion extendable from said aperture.

9. The intravascular treatment device of claim 1, wherein the deployment member comprises a tubular delivery member and at least one branch lead extends from said branch vessel flow element and is threadingly engageable with the exterior of said tubular delivery member.

10. The intravascular treatment device of claim 1, wherein an anchoring stent is provided for sealing engagement with said branch vessel flow element and a wall of a branch vessel within which said branch vessel exclusion element may be deployed.

11. An intravascular treatment device for excluding an aneurysmal site in a body flow lumen wherein the flow lumen includes branch flow lumens extending therefrom, comprising:
a catheter containing:
a tubular exclusion portion positionable to span the aneurysmal site and including at least one aperture therewith positionable adjacent to a branch flow lumen;
a branch vessel flow element integral with said tubular exclusion portion, said branch vessel flow element being extendable from said aperture and having a distal end distant from said aperture; and
a branch lead releasably connected to said distal end of said element and threaded to the exterior of said catheter.

12. The intravascular treatment device of claim 11, wherein said branch vessel flow element includes an extending portion extendable from, and in sealing engagement with, a flange sealingly engaged to said tubular extension portion at said at least one aperture.

13. The intravascular treatment device of claim 12, wherein said branch vessel flow element includes a distal end portion extendable from a position adjacent to said tubular exclusion portion to a position spaced from said tubular exclusion portion.

14. The intravascular treatment device of claim 13, further including at least one branch lead releasably connected to said distal end of said branch flow element.

15. The intravascular treatment device of claim 14, wherein said distal end portion includes at least one aperture therethrough, and said branch lead extends through said aperture.

16. The intravascular treatment device of claim 11, further including an anchoring stent configured for sealing engagement with said branch flow element and with the interior of a branch flow lumen within which at least a portion of said branch flow element may be located.

17. An intravascular treatment device for excluding an aneurysmal site in a body flow lumen wherein the flow lumen include sat least one branch lumen and the treatment device includes at least one aperture therein which is configurable to access the branch lumen, comprising:
a tubular portion having opposed ends positionable in sealing engagement with said body lumen upstream and downstream of the aneurysmal site and having the aperture located through the tubular portion at a location intermediate of said ends; and
a branch flow portion enagagable with said aperture and extendable therefrom into said branch lumen, said branch flow portion including a first portion in sealing engagement with said tubular portion adjacent to said aperture and a second portion extendable therefrom, said second portion incapable of sealing engagement with said tubular portion.

18. The intravascular treatment vessel of claim 17, wherein said second portion has insufficient rigidity to maintain a tubular profile upon deployment in a body flow lumen.

19. The intravascular treatment device of claim 17, further including an anchoring element for anchoring said second portion in a branch vessel in a tubular open profile.
